SEQUENCE LISTING (4 sequences)

SEQ ID NO: 1 - DNA, 1932 bp, Human immunodeficiency virus type 1
gggaacttgtgggtcacagtctattatggggtacctgtgtggaaagaagcaaaaactactctattctgtgcatcagatgctaaatcatatgagaaagaagtgcataatgtctgggctacacatgcctgtgtacccacagaccccaacccacaagaaatagttttgggaaatgtaacagaaaattttaacatgtggaaaaatgacatggtggatcagatgcatgaggatataatcagtttatgggatcaaagcctaaagccatgtgtaaagttgaccccactctgtgtcactttaaattgtacagaggttaatgttaccagaaatgttaataatagcgtggttaataataccacaaatgttaataatagcatgaatggagacatgaaaaattgctctttcaacataaccacagaactaaaagataagaaaaagaatgtgtatgcacttttttataaacttgatatagtatcacttaatgagactgacgactctgagactggcaactctagtaaatattatagattaataaattgtaatacctcagccctaacacaagcctgtccaaaggtctcttttgacccaattcctatacattattgtgctccagctggttatgcgattctaaagtgtaataataagacattcaatgggacaggaccatgccataatgtcagcacagtacaatgtacacatggaattaagccagtggtatcaactcaactactgttaaatggtagcctagcagaagaagggataataattagatctgaaaatctgacaaacaatgtcaaaacaataatagtacatcttaatagatctatagaaattgtgtgtgtaagacccaacaataatacaagacaaagtataagaataggaccaggacaaacattctatgcaacaggagacataataggagacataagacaagcacattgtaacattagtaggactaactggactaagactttacgagaggtaaggaacaaattaagagaacacttccctaataaaaacataacatttaaaccatcctcaggaggggacctagaaattacaacacatagctttaattgtagaggagaatttttctattgcaatacatcgggcctgtttagtataaattatacagaaaataatacagatggtacacccatcacactcccatgcagaataagacaaattataaatatgtggcaggaagtaggacgagcaatgtacgcccctcccattgaaggaaacatagcatgtaaatcagatatcacagggctactattggttcgggatggaggaagcacaaatgatagcacaaataataacacagagatattcagacctgcaggaggagatatgagggacaattggaggagtgaattgtataagtataaagtggtagaaattaagccattgggaatagcacccactgaggcaaaaaggagagtggtggagagagaaaaatcagcagtgggaataggagctgtgttccttgggttcttgggagcagcaggaagcactatgggcgcagcgtcaataacgctgacggcacaggccagacaagtgttgtctggtatagtgcaacagcaaagcaatttgctgagggctatagaggcgcaacagcatctgttgcaactcacggtctggggcattaagcagctccagacaagagtcctggctatagaaagatacctaaaggatcaacagctcctaggactttggggctgctctggaaaactcatctgcaccactgctgtgccttggaacatcagttggagtaataaatctaaaacagatatttgggataacatgacctggatgcagtgggatagagaaattagtaattacacaaacacaatatacaggttgcttgaggactcgcagagccagcaggagcaaaatgaaaaagatttattagcattggacagttggaacaattga

SEQ ID NO: 2 - protein, 643 aa, Human immunodeficiency virus type 1
GNLWVTVYYGVPVWKEAKTTLFCASDAKSYEKEVHNVWATHACVPTDPNPQEIVLGNVTENFNMWKNDMVDQMHEDIISLWDQSLKPCVKLTPLCVTLNCTEVNVTRNVNNSVVNNTTNVNNSMNGDMKNCSFNITTELKDKKKNVYALFYKLDIVSLNETDDSETGNSSKYYRLINCNTSALTQACPKVSFDPIPIHYCAPAGYAILKCNNKTFNGTGPCHNVSTVQCTHGIKPVVSTQLLLNGSLAEEGIIIRSENLTNNVKTIIVHLNRSIEIVCVRPNNNTRQSIRIGPGQTFYATGDIIGDIRQAHCNISRTNWTKTLREVRNKLREHFPNKNITFKPSSGGDLEITTHSFNCRGEFFYCNTSGLFSINYTENNTDGTPITLPCRIRQIINMWQEVGRAMYAPPIEGNIACKSDITGLLLVRDGGSTNDSTNNNTEIFRPAGGDMRDNWRSELYKYKVVEIKPLGIAPTEAKRRVVEREKSAVGIGAVFLGFLGAAGSTMGAASITLTAQARQVLSGIVQQQSNLLRAIEAQQHLLQLTVWGIKQLQTRVLAIERYLKDQQLLGLWGCSGKLICTTAVPWNISWSNKSKTDIWDNMTWMQWDREISNYTNTIYRLLEDSQSQQEQNEKDLLALDSWNN

SEQ ID NO: 3 - DNA, 1902 bp, Human immunodeficiency virus type 1
ggaaacttgtgggtcacagtctattatggggtacctgtatggaaaggggcaaccaccactttattttgtgcatcagatgctaaagcatatgatacagaggtacataatgtttgggctacacatgcctgtgtacccgcagaccccaacccacaagaaatggttttggaaaatgtaacagaaaattttaacatgtggaaaaatgaaatggtaaatcagatgcaggaagatgtaatcagtttatgggatcaaagcctaaaaccatgtgtaaagttgaccccactctgtgtcactttagaatgtagaaatgttagcagtaatagtaatgatacctaccatgagacctaccatgagagcatgaaggaaatgaaaaattgctctttcaatgcaaccacagtagtaagagataggaagcagacagtgtatgcacttttttatagacttgatatagtaccacttactaagaagaactatagtgagaattctagtgagtattatagattaataaattgtaatacctcagccataacacaagcctgtccaaaggtcacttttgatccaattcctatacactattgcactccagctggttatgcaattctaaagtgtaatgataagatattcaatgggacaggaccatgccataatgttagcacagtacaatgtacacatgggattaagccagtggtatcaactcaactactgttaaatggtagcctagcagaaggagaaataataattagatctgaaaatctgacaaacaatgtcaaaacaataatagtacatcttaatcaatctgtagaaattgtatgtacaagacccggcaataatacaagaaaaagtataaggataggaccaggacaaacattctatgcaacaggagacataataggagacataagacaagcacattgtaacattagtgaagataaatggaatgaaactttacaaagggtaagtaaaaaattagcagaacacttccagaataaaacaataaaatttgcatcatcctcaggaggggacctagaagttacaacacatagctttaattgtagaggagaatttttctattgtaatacatcaggcctgtttaatggtgcatacacgcctaatggtacaaaaagtaattcaagctcaatcatcacaatcccatgcagaataaagcaaattataaatatgtggcaggaggtaggacgagcaatgtatgcccctcccataaaaggaaacataacatgtaaatcaaatatcacaggactactattggtacgtgatggaggaacagagccaaatgatacagagacattcagacctggaggaggagatatgaggaacaattggagaagtgaattatataaatataaagtggtagaaattaagccattgggagtagcacccactacaacaaaaaggagagtggtggagagagaaaaaagagcagtgggaataggagctgtgttccttgggttcttaggagtagcaggaagcactatgggcgcggcgtcaataacgctgacggtacaggccagacaattgctgtctggtatagtgcaacagcaaagcaatttgctgagggctatagaagcgcaacagcatctgttgcaactcacggtctggggcattaagcagctccagacaagagtcctggctatagaaagatacctaaaggatcaacagctcctagggatttggggctgctctggaaaactcatctgcactactgctgtaccttggaactccagttggagtaacaaatctcaaaaagagatttgggataacatgacctggatgcaatgggataaagaaattagtaattacacaaacacagtatacaggttgcttgaagaatcgcaaaaccagcaggaaaggaatgaaaaagatctattagcattggacagttggaaaaat

SEQ ID NO: 4 - protein, 634 aa, Human immunodeficiency virus type 1
GNLWVTVYYGVPVWKGATTTLFCASDAKAYDTEVHNVWATHACVPADPNPQEMVLENVTENFNMWKNEMVNQMQEDVISLWDQSLKPCVKLTPLCVTLECRNVSSNSNDTYHETYHESMKEMKNCSFNATTVVRDRKQTVYALFYRLDIVPLTKKNYSENSSEYYRLINCNTSAITQACPKVTFDPIPIHYCTPAGYAILKCNDKIFNGTGPCHNVSTVQCTHGIKPVVSTQLLLNGSLAEGEIIIRSENLTNNVKTIIVHLNQSVEIVCTRPGNNTRKSIRIGPGQTFYATGDIIGDIRQAHCNISEDKWNETLQRVSKKLAEHFQNKTIKFASSSGGDLEVTTHSFNCRGEFFYCNTSGLFNGAYTPNGTKSNSSSIITIPCRIKQIINMWQEVGRAMYAPPIKGNITCKSNITGLLLVRDGGTEPNDTETFRPGGGDMRNNWRSELYKYKVVEIKPLGVAPTTTKRRVVEREKRAVGIGAVFLGFLGVAGSTMGAASITLTVQARQLLSGIVQQQSNLLRAIEAQQHLLQLTVWGIKQLQTRVLAIERYLKDQQLLGIWGCSGKLICTTAVPWNSSWSNKSQKEIWDNMTWMQWDKEISNYTNTVYRLLEESQNQQERNEKDLLALDSWKN

(19) United States
(12) Patent Application Publication
(10) Pub. No.: US 2009/0297551 A1
(43) Pub. Date: Dec. 3, 2009

(54) ADJUVANT

(75) Inventors: Quentin Sattentau, Oxford (GB); Neil Sheppard, Oxford (GB)

Correspondence Address: FENWICK & WEST LLP, SILICON VALLEY CENTER, 801 CALIFORNIA STREET, MOUNTAIN VIEW, CA, 94041, US

(73) Assignee: ISIS INNOVATION LIMITED, Oxford (GB)

(21) Appl. No.: 12/293,380

(22) PCT Filed: Mar. 19, 2007
(86) PCT No.: PCT/GB2007/000979

§ 371 Date: Apr. 9, 2009

(30) Foreign Application Priority Data
Mar. 18, 2006 (GB) 0605521.4

(51) Int. Cl. A61K 39/00; A61K 47/06; A61K 45/00; A61K 39/12; A61K 39/21; A61K 39/245; A61K 39/095; A61K 39/118; A61K 39/02; A61P 37/04

(52) U.S. Cl. 424/192.1; 424/280.1; 424/278.1; 424/184.1; 424/204.1; 424/208.1; 424/231.1; 424/249.1; 424/263.1; 424/274.1; 424/234.1

(57) ABSTRACT

This invention relates to a novel adjuvant comprising a transfection reagent, and to uses of this adjuvant. In particular, the adjuvant may be used in compositions for eliciting an immune response and in vaccines.

This invention relates to a novel adjuvant composition, to uses of the adjuvant composition and to vaccine compositions including the adjuvant.

When a human or non-human animal is challenged by a foreign organism/pathogen the challenged individual responds by launching an immune response which may be protective. This immune response is characterised by the co-ordinated interaction of the innate and acquired immune response systems.

The innate immune response forms the first line of defense against a foreign organism/pathogen. An innate immune response may be triggered within minutes of infection in an antigen-independent, but pathogen-dependent, manner. The innate, and indeed the adaptive, immune system can be triggered by the recognition of pathogen associated molecular patterns (PAMPs) unique to microorganisms by pattern recognition receptors (PRR) present on most host cells. Once triggered the innate system generates an inflammatory response that activates the cellular and humoral adaptive immune response systems.

The adaptive immune response becomes effective over days or weeks and provides the antigen specific responses needed to control and usually eliminate the foreign organism/pathogen. The adaptive response is mediated by T cells (cell mediated immunity) and B cells (antibody mediated or humoral immunity) that have developed specificity for the pathogen. Once activated these cells have a long lasting memory for the same pathogen.

The ability of an individual to generate immunity to foreign organisms/pathogens, thereby preventing or at least reducing the chance of infection by the foreign organism/pathogen, is a powerful tool in disease control and is the principle behind vaccination.

Vaccines function by preparing the immune system to mount a response to a pathogen. Typically, a vaccine comprises an antigen, which is a foreign organism/pathogen or a toxin produced by an organism/pathogen, or a portion thereof, that is introduced into the body of a subject to be vaccinated in a non-toxic, non-infectious and/or non-pathogenic form. The antigen in the vaccine causes the subject's immune system to be “primed” or “sensitised” to the organism/pathogen from which the antigen is derived. Subsequent exposure of the immune system of the subject to the organism/pathogen or toxin results in a rapid and robust immune response, that controls or destroys the organism/pathogen or toxin before it can multiply and infect or damage enough cells in the host organism to cause disease symptoms.

In many cases it is necessary to enhance the immune response to the antigens present in a vaccine in order to stimulate the immune system to a sufficient extent to make a vaccine effective, that is, to confer immunity. To this end, additives known as adjuvants (or immune potentiators) have been devised which enhance the in vivo immune response to an antigen in a vaccine composition.

An adjuvant composition increases the strength and/or duration of an immune response to an antigen relative to that elicited by the antigen alone. A desired functional characteristic of an adjuvant composition is its ability to enhance an appropriate immune response to a target antigen.

Known adjuvant compositions include oil emulsions (Freund's adjuvant), oil based compounds (i.e. MF59), saponins, aluminium or calcium salts (i.e. Alum), non-ionic block polymer surfactants, lipopolysaccharides (LPS), attenuated or killed mycobacteria, tetanus toxoid and others.

Until very recently aluminium salt (Alum) was the only adjuvant licensed for vaccine use in humans. More recently the oil-based adjuvant MF59 and virosomes have also received FDA approval for vaccine use in humans (Pashine et al, Nature Medicine 11: S63-68 (2005)).

The human immunodeficiency virus (HIV) is an example of where an adjuvant appears to be needed in order to develop a vaccine. Antigens derived from HIV have to date not been successfully used as vaccines. The administration to an individual of the HIV type-1 (HIV-1) envelope glycoprotein (Env), or parts thereof, as a vaccine have not been able to induce a sufficient immune response to confer immunity on the individual. The poor immunogenicity of HIV-1 Env, and indeed HIV type-2 (HIV-2) Env and simian immunodeficiency virus (SIV) Env, may be due to factors such as the infrequency of helper T-cell epitopes on the Env antigens from some strains, the extensive glycosylation of the Env protein, and even the fact that the native Env structure itself may serve to restrict optimal proteolytic processing.

According to a first aspect, the invention provides the use of a transfection reagent as an adjuvant.

According to another aspect, the invention provides an adjuvant composition comprising a transfection reagent.

A transfection reagent is a composition that allows molecules, including proteins and/or nucleic acids, to move across the limiting lipid cell membrane (the plasma membrane) of animal cells, for example human cells, and into the cell cytoplasm.

Preferably the transfection reagent is non-liposomal. Non-liposomal transfection reagents may comprise lipids in a form such as cationic polymers.

The transfection reagent may be a cationic polymer. Cationic polymers may bind the anionic outer surface of a cell membrane.

Alternatively, or additionally, non-liposomal transfection reagents may comprise agents such as virosomes, virosomes may use fusion proteins to fuse with the plasma membrane.

The non-liposomal transfection reagent may be selected from FuGENE 6™ (a non-liposomal multicomponent reagent available form Roche Diagnostics Ltd.), polyetheylenimine (PEI available from Sigma Aldrich), effective derivatives of PEI both linear and branched, cationic polymers, polybrene, monovalent cationic lipids such as DOTMA, DOTAP and LHON (Zhang et al, J. Controlled Release 100: 165-180 (2004)), cationic triglycerides, polyvalent cationic lipids such as DOGS, DOSPA, DPPES and natural glycine betaines (GBs) (Zhang et al, J. Controlled Release 100: 165-180 (2004)), guanidine-containing compounds, cationic peptides including poly-L-Lysine and protamine (Zhang et al, J. Controlled Release 100: 165-180 (2004)) or a combination thereof.

Non-liposomal transfection reagents are cheap and easy to make, and less likely to cause damage to an antigen and/or a ligand than a liposomal transfection reagent.

Preferably the transfection reagent is PEI. PEI is known for use as a transfection reagent both in vitro and in vivo. PEI is a potent transfection reagent, which is approximately 10,000-fold more efficient than poly-L-lysine. Under optimal conditions the transfection efficiency of PEI is similar to viral vectors

Preferably PEI is uncomplexed. Preferably PEI has a high cationic charge density.

Preferably PEI has a molecular weight of between about 1000 Da and about 1600 kDa. Preferably PEI has a molecular weight of between about 1 kDa and about 100 kDa, more preferably PEI has a molecular weight of between about 1 kDa and about 50 kDa, preferably between about 5 kDa and about 25 kDa, preferably about 25 kDa.

The PEI used may be branched or linear, or a combination thereof.

The transfection reagent may be a PEI-based polymer.

According to a further aspect the invention provides the use of PEI as an adjuvant.

According to a yet further aspect the invention provides an adjuvant comprising PEI.

Preferably an adjuvant according to any aspect of the invention is for use as part of a composition which elicits an immune response when administered. The composition may also comprise one or more antigens. Preferably the composition is a vaccine composition.

An adjuvant composition according to any aspect of the invention may be used with any suitable antigen.

In use the adjuvant and antigen may be administered simultaneously, sequentially or separately.

In use, the adjuvant and antigen may be in the same or different compositions.

The antigen may be a nucleic acid, a protein, a peptide, a glycoprotein, a polysaccharide or other carbohydrate, a fusion protein, a lipid, a glycolipid, a peptide mimic of a polysaccharide, a cell or a cell extract, a dead or attenuated cell or extract thereof, a tumour cell or an extract thereof, or a viral particle or an extract thereof, or any combination thereof.

The antigen may be derived from a human or non-human animal, a bacterium, a virus, a fungus, a protozoan or a prion.

Preferably the antigen is derived from a pathogen, such as a virus, a bacterium or a fungus. For example, the antigen may be a protein or polypeptide derived from one or more of the following pathogens, HIV type 1 and 2 (HIV-1 and HIV-2 respectively), the Human T Cell Leukaemia Virus types 1 and 2 (HTLV-1 and HTLV-2 respectively), the Herpes Simplex Virus types 1 and 2 (HSV-1 and HSV-2 respectively), human papilloma virus, Treponema pallidum, Neisseria gonorrhoea, Chlamydia trachomatis and Candida albicans.

The antigen may be naturally produced (e.g. purified from the pathogen), recombinantly produced (e.g. from a genetically-engineered expression system) or a synthetic product. The antigen may be a modified form of a natural product, for example the antigen may include modifications such as deletions, insertions, additions and substitutions, so long as the antigen elicits an immunological response that would recognise both the modified and the natural product.

Preferably the antigen is a protein, or a part of a protein, derived from HIV-1 or HIV-2. Preferably the antigen is an HIV envelope glycoprotein (Env), or a fragment or an immunogenic derivative thereof. Preferably the protein is the HIV envelope glycoprotein gp140 or a fragment or an immunogenic derivative thereof, or a peptide or small molecule that mimics an antigenic epitope of the HIV envelope glycoprotein gp140.

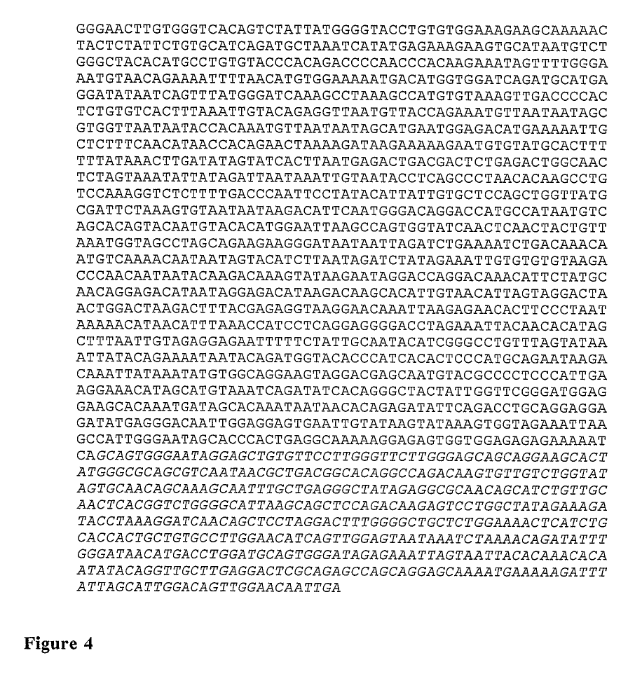

Preferably the antigen is selected from the group comprising the proteins HIV-1_{zm96}gp140, HIV-1_{IIIB}gp140 and HIV-1_{CN54}gp140 or a fragment or immunogenic derivative thereof. Preferably, the antigen is the HIV-1_{zm96}gp140 protein as encoded by the sequence of Sequence ID No. 1 (FIG. 4) or by a nucleic acid molecule comprising a sequence which is a variant of Sequence ID No. 1 having at least 65% identity to the sequence of Sequence ID No. 1. The nucleic acid sequence preferably has at least 70%, 75% or 80% identity to Sequence ID No. 1. Even more preferably, the nucleic acid sequence has 85%, 90%, 95%, 98%, 99%, 99.9% or even higher identity to Sequence ID No. 1.

Preferably, when the protein encoded by a nucleic acid sequence that has at least 65% identity to Sequence ID No. 1 is administered to a host it will elicit an immune response that will also recognise the protein encoded by Sequence ID No. 1.

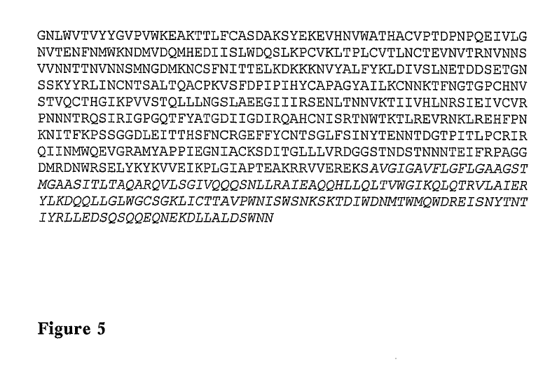

Preferably, the antigen is a HIV-1_{zm96}gp140 protein, or an antigen derived from HIV-1_{zm96}gp140, having the sequence of Sequence ID No. 2 (FIG. 5) or a protein comprising a sequence which is a variant of Sequence ID No. 2 having at least 65% identity to the sequence of Sequence ID No. 2. The protein preferably has at least 70%, 75% or 80% identity to Sequence ID No. 2. Even more preferably, the protein has 85%, 90%, 95%, 98%, 99%, 99.9% or even higher identity to Sequence ID No. 2.

Preferably, when a protein with at least 65% identity to Sequence ID No. 2 is administered to a host it will elicit an immune response that will also recognise the protein of Sequence ID No. 2.

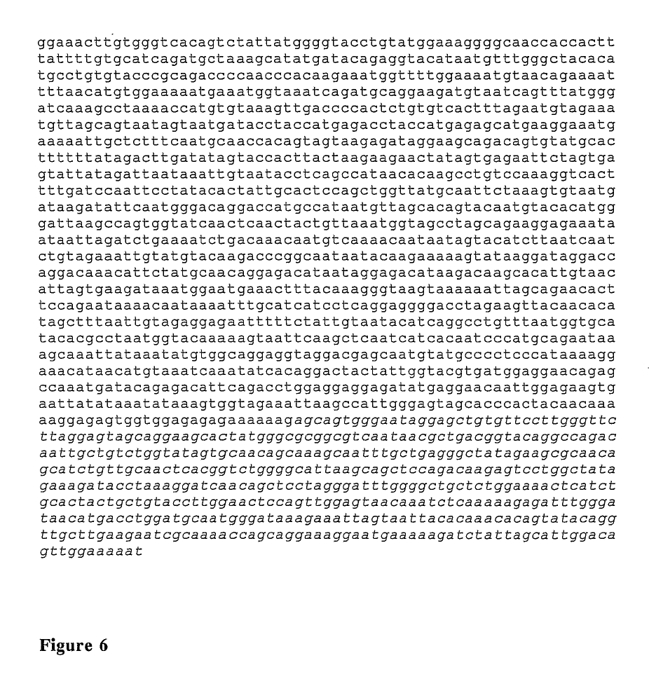

Preferably, the antigen is the HIV-1_{CN54}gp140 protein as encoded by the sequence of Sequence ID No. 3 (FIG. 6) or by a nucleic acid molecule comprising a sequence which is a variant of Sequence ID No. 3 having at least 65% identity to the sequence of Sequence ID No. 3. The nucleic acid sequence preferably has at least 70%, 75% or 80% identity to Sequence ID No. 3. Even more preferably, the nucleic acid sequence has 85%, 90%, 95%, 98%, 99%, 99.9% or even higher identity to Sequence ID No. 3.

Preferably, when the protein encoded by a nucleic acid sequence that has at least 65% identity to Sequence ID No. 3 is administered to a host it will elicit an immune response that will also recognise the protein encoded by Sequence ID No. 3.

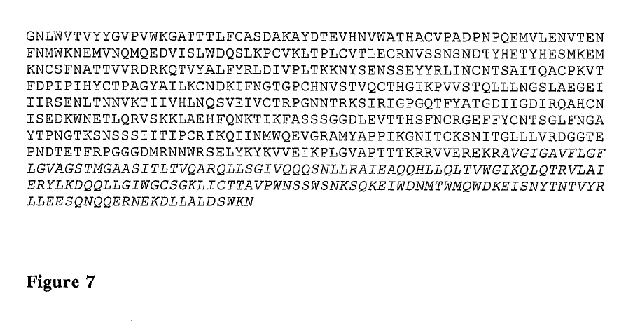

Preferably, the antigen is a HIV-1_{CN54}gp140 protein, or a protein derived from HIV-1_{CN54}gp140, having the sequence of Sequence ID No. 4 (FIG. 7) or a protein comprising a sequence which is a variant of Sequence ID No. 4 having at least 65% identity to the sequence of Sequence ID No. 4. The protein preferably has at least 70%, 75% or 80% identity to Sequence ID No. 4. Even more preferably, the protein has 85%, 90%, 95%, 98%, 99%, 99.9% or even higher identity to Sequence ID No. 4.

Preferably, when a protein with at least 65% identity to Sequence ID No. 4 is administered to a host it will elicit an immune response that will also recognise the protein of Sequence ID No. 4.

The term “identity” in the context of nucleic acid and protein sequences refers to the residues in the two sequences which are the same when the sequences are aligned for maximum correspondence. The length of sequence identity comparison may be over a stretch of at least about nine nucleotides, usually at least about 20 nucleotides/amino acids, more usually at least about 24 nucleotides/amino acids, typically at least about 28 nucleotides, more typically at least about 32 nucleotides/amino acids, and preferably at least about 36 or more nucleotides/amino acids. There are a number of different algorithms known in the art which can be used to measure nucleotide/amino acid sequence identity. For instance, polynucleotide sequences can be compared using FASTA, Gap or Bestfit, which are programs in Wisconsin Package Version 10.0, Genetics Computer Group (GCG), Madison, Wis. FASTA provides alignments and percent sequence identity of the regions of the best overlap between the query and search sequences (Pearson, Methods Enzymol. 183:63-98 (1990)). For instance, percent sequence identity between nucleic acid sequences can be determined using FASTA with its default parameters (a word size of 6 and the NOPAM factor for the scoring matrix) or using Gap with its default parameters as provided in GCG Version 6.1. Alternatively, sequences can be compared using the computer program, BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

Alternatively, the antigen may be another form of an HIV envelope protein for example, gp160 or gp120, or a fragment or an immunogenic derivative thereof.

The composition may comprise more than one antigen derived from the same or different pathogens.

The adjuvant composition may, in use, stimulate a Th₁ (type 1—a cytotoxic T cell response) immune response.

Alternatively, the adjuvant composition may, in use, stimulate a Th₂ (type 2—a B cell antibody response) immune response.

Alternatively, the adjuvant composition may, in use, stimulate a Th₁ and a Th₂ immune response.

The adjuvant composition may also comprise one or more ligands for one or more intracellular immune response receptors.

Preferably the intracellular immune response receptor is an innate immune response receptor.

An intracellular immune response receptor refers to a receptor which when activated by the binding of a ligand triggers a response associated with the immune response. Preferably, the response is associated with the innate immune system. Examples of intracellular immune response receptors and their ligands include, Toll-Like Receptor (TLR)-9 which is found in an endocytic compartment within cells and which responds to viral and intracellular bacterial unmethylated DNA that is rich in CpG sequences. Another example is TLR-3, also found in an endocytic compartment, which responds to viral double-stranded RNA or the analogue, poly I:C (Kopp and Medzhitov, Curr. Opp. in Immunol. 15: 396-401 (2003) and Janssens and Beyaert, Clin. Microb. Rev. 16: 637-646 (2003)). A third example is the cellular cytoplasmic enzyme RNA-dependent protein kinase (PKR), that is activated by viral RNA acid in the cytoplasm and leads to interferon production and cell apoptosis (Malmgaard, J. Interferon. Res. 24: 439-454 (2004)).

A ligand for use in the invention may be for an intracellular innate immune response receptor selected from the group comprising TLR3, TLR7, TLR8, TLR9, NOD1, NOD2, RIG1, RIG2, MDA-5 and PKR. Preferably the ligand is for a Toll-Like Receptor, more preferably for TLR3, TLR7, TLR8 and/or TLR9.

The ligand may be a nucleic acid. The ligand may be CpG-ODN. CpG-ODN is known to stimulate immune activation through the Toll-Like Receptor-9 (TLR-9). Preferably the backbone of the CpG-ODN has been modified to produce phosphorothioate rather than natural phosphodiester DNA molecules. This modification enables the CpG-ODN to resist attack by nucleases.

The ligand may be single or double stranded RNA or DNA molecule. The ligand may be polyriboinosinic polyribocytidylic acid (Poly(I:C))—a double stranded RNA mimetic. Poly(I:C) is known to stimulate immune activation through the Toll-Like Receptor-3 (TLR-3). Alternatively, the ligand may be an imidazoquinoline such as imiquimod (for example, Resiquimod™ from 3M), which mimics single stranded RNA. Single stranded RNA is known to stimulate immune activation through the Toll-Like Receptors-7 and/or 8 (TLR-7/8).

The receptors TLR3 and TLR9 are endosomally located, and thus a ligand for these receptors has to pass through the plasma membrane and enter the endosome. A non-liposomal transfection reagent may help in this process.

The adjuvant composition may contain one or more TLR ligands; the one or more ligands may target the same or different TLRs.

The Toll Like Receptors (TLRs) are a highly conserved family of PRRs and are related to the receptor Toll, characterised in Drosophila melanogaster. Eleven TLRs have been identified to date, with the majority conserved in mouse and man, however the cell types that express these TLRs is know to vary to some extent. For example, TLR9 is expressed in plasmacytoid dendritic cells (pDCs), B-cells, NK cells and monocytes in man (Bauer et al (2001) PNAS 98(16) 9237-9242; Hornung et al (2002) J Immunol 168(9) 4531-4537; Gursel et el (2002) J Leukoc Biol 71(5) 813-820), but is found in both myeloid and plasmacytoid dendritic cells in mice as well as in B-cells, NK cells and monocytes (Kreig A M (2002) Annu Rev Immunol 20 709-760). TLR ligands are potent activators of the innate and adaptive immune responses and therefore have been considered potential adjuvants for vaccine use. TLRs 3, 7, 8 and 9 are found in an intracellular compartment, and it is necessary for their ligands (for example, dsRNA for TLR3, ssRNA, R-837, R848, loxoribine or bropirimine for TLR7, ssRNA or R848 for TLR8 and CpG-ODN for TLR9 (Akira & Takeda (2004) Nat Rev Immunol 4(7) 499-511) to enter this compartment in order to trigger receptor signalling and immune activation (Matsumoto et al (2003) 171(6) 3154-3162; Roman et al (1997 3(8) 849-854).

Preferably the only active component of the adjuvant is a transfection reagent, such as PEI.

Preferably the adjuvant does not comprise a ligand for one or more intracellular immune response receptors.

According to a further aspect the invention comprises an immunogenic composition capable of eliciting an immune response to an antigen when administered to a human or non-human animal, said immunogenic composition comprising an adjuvant composition according to the invention and one or more antigens. The immunogenic composition may be a vaccine.

The antigen may be an antigen as described above.

The adjuvant composition, or immunogenic composition, may also comprise one or more auxiliary adjuvants. The auxiliary adjuvant may stimulate a Th₁ and/or a Th₂ immune response. The auxiliary adjuvant may be any other effective adjuvant, for example, Alum or MF59.

Preferably the adjuvant, or immunogenic composition, does not comprise a ligand for one or more intracellular immune response receptors.

Preferably the adjuvant composition, or immunogenic composition, is for use in therapeutic or prophylactic treatments or both.

Preferably the adjuvant composition, or immunogenic composition, is for use in immune activation and/or modulation. The adjuvant may be used in a composition to stimulate an immune response, for example, in a vaccine composition or in an antiviral or anticancer composition or drug. The adjuvant may also be used in a composition to modulate or control an immune response, for example, in a composition to control an allergic reaction. The adjuvant composition may be used alone, without a specific antigen, to control an immune response. The antigen may be an environmental antigen, such as pollen, nuts or other allergens. To control an allergic reaction the adjuvant composition may stimulate a Th₁ response. Alternatively, or in addition, the composition may stimulate a Th₂ response.

According to another aspect, the invention provides the use of an adjuvant composition according to the invention in the preparation of a composition for eliciting an immune response. The composition for eliciting an immune response may be a vaccine.

A composition for eliciting an immune response according to this aspect of the invention may also comprise one or more antigens.

The optimal ratios of each component in a composition according to the invention may be determined by techniques well known to those skilled in the art. The composition for eliciting an immune response preferably contains a therapeutically and/or a prophylactically effective amount of an antigen and an adjuvant composition. An effective amount of antigen and adjuvant is preferably an amount sufficient to cause an immune response in the host. Preferably the immune response effected by a composition to be used as a vaccine is protective and is enough to reduce or prevent infection of the vaccinated host by the pathogen from which the antigen is derived or is based.

A vaccine composition, or a composition for eliciting an immune response, according to the invention may comprise about 10 μM non-liposomal transfection reagent and about 0.06% antigen. The composition may optionally also comprise about 100 μg of a ligand for one or more intracellular immune response receptors in about 100 μl (that is, about 0.1% ligand). The ligand may be CpG. In another embodiment, a composition of 100 μl preferably comprises about 0.25% of a non-liposomal transfection reagent and about 0.06% antigen. The composition may optionally comprise about 0.1% ligand for one or more intracellular immune response receptors. The non-liposomal transfection reagent may be PEI.

According to a further aspect the invention provides the use of a transfection reagent as an adjuvant in the preparation of a composition for eliciting an immune response. Preferably the transfection reagent is non-liposomal. Preferably the composition is a vaccine. Preferably the composition also comprises one or more antigens. Preferably the composition comprises one or more ligands to one or more intracellular immune response receptors. Preferably the composition does not comprise one or more ligands to one or more intracellular immune response receptors.

According to a yet further aspect, the invention provides a vaccine composition comprising an adjuvant according to the invention and one or more antigens.

According to a yet further aspect, the invention provides an antiviral and/or an anti-cancer and/or an immuno-modulating composition comprising an adjuvant according to the invention and one or more antigens.

Preferably the vaccine composition, the antiviral, the anti-cancer and/or the immuno-modulating composition according to the invention does not comprise one or more ligands to one or more intracellular immune response receptors.

A vaccine composition, or a composition for eliciting an immune response, according to the invention may be for oral, systemic, parenteral, topical, mucosal, intramuscular, intradermal, subcutaneous, intranasal, intravaginal, sublingual, or inhalation administration.

Preferably, a vaccine composition, or a composition for eliciting an immune response, according to the invention is intended for administration to a human.

A composition according to the invention may be administered to a subject in the form of a pharmaceutical composition. A pharmaceutical composition preferably comprises one or more physiologically effective carriers, diluents, excipients or auxiliaries which facilitate processing and/or delivery of the antigen and/or adjuvant.

Determination of an effective amount of a vaccine composition, or a composition for eliciting an immune response, for administration is well within the capabilities of those skilled in the art. In general the amount of antigen in a dose of a vaccine composition ranges from about 1 pg to about 100 mg per kg of host, typically from about 10 pg to about 1 mg per dose, more preferably about 1 ng to about 100 μg per dose, more preferably about 1 μg to about 100 μg per dose.

Preferably the active ingredients in a composition according to the invention are greater than 50% pure, usually greater than 80% pure, often greater than 90% pure and more preferably greater than 95%, 98% or 99% pure. With active compounds approaching 100% pure, for example about 99.5% pure or about 99.9% pure, being used most often.

According to another aspect, the invention provides an immunogenic composition capable of eliciting an immune response when administered to a human or non-human animal comprising an adjuvant according to the invention. Preferably the immunogenic composition also comprises one or more antigens.

The non-human animal may be a mammal, bird or fish.

According to yet another aspect, the invention provides a method for inducing or enhancing immunogenicity of an antigen in a human or non-human animal to be treated comprising administering to said subject one or more antigens and an adjuvant composition according to the invention in an amount effective to induce or enhance the immunogenicity of the antigen in the subject. The adjuvant and antigen may be administered simultaneously, sequentially or separately.

Preferably the method produces an immune reaction sufficient to vaccinate a subject against a pathogen from which the antigen is derived.

The subject may be a human or non-human animal, including mammals, birds and fish.

The skilled man will appreciate that any of the preferable/optional features discussed above can be applied to any of the aspects of the invention.

The terms adjuvant and adjuvant composition are intended to have the same meaning and are used interchangeably. Similarly, the terms vaccine and vaccine composition are intended to have the same meaning and are used interchangeably.

Preferred embodiments of the present invention will now be described, merely by way of example, with reference to the following drawings and examples.

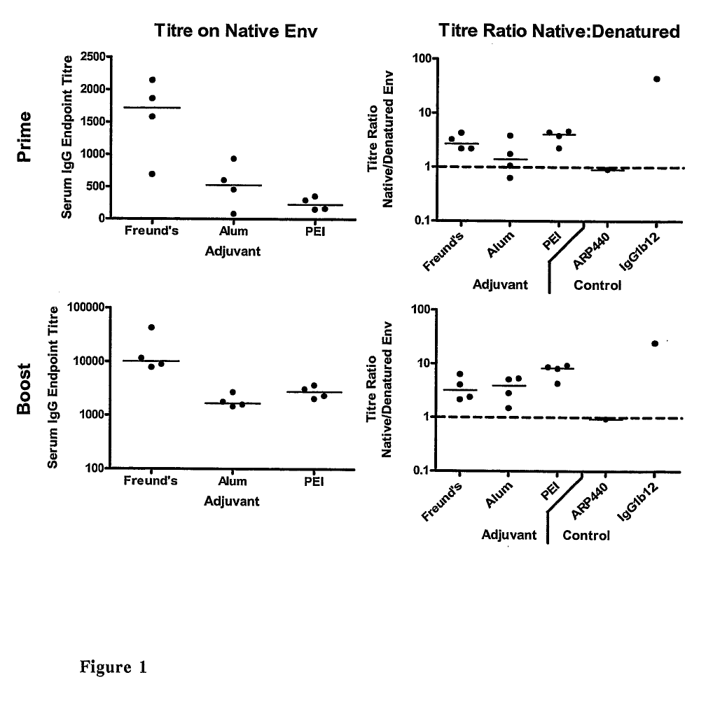

FIG. 1—shows the results of a comparative adjuvant study in rabbits. More specifically, FIG. 1 illustrates the endpoint antibody titre following an initial prime and after a boost in rabbits immunised with the antigen HIV-1_{zm96}gp140 (gp140_{zm96}) and an adjuvant selected from Freunds adjuvant, alum and PEI;

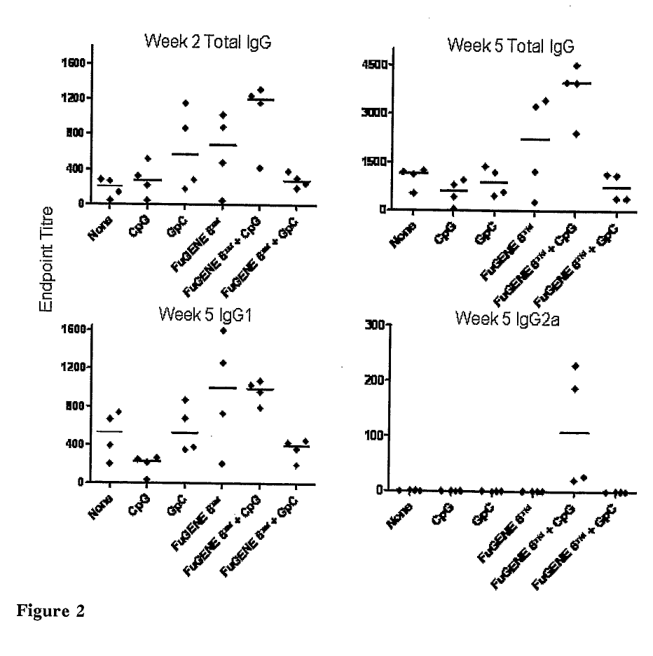

FIG. 2—shows the results of a comparative adjuvant study in mice. More specifically, FIG. 2 illustrates the endpoint antibody titres two or five weeks after a single immunisation for mice immunised with the antigen HIV-1_{CN54}gp140 (gp140_{CN54}) and an adjuvant composition comprising the transfection reagent FuGENE 6™ and/or the TLR ligand CpG-ODN. Control data are also shown;

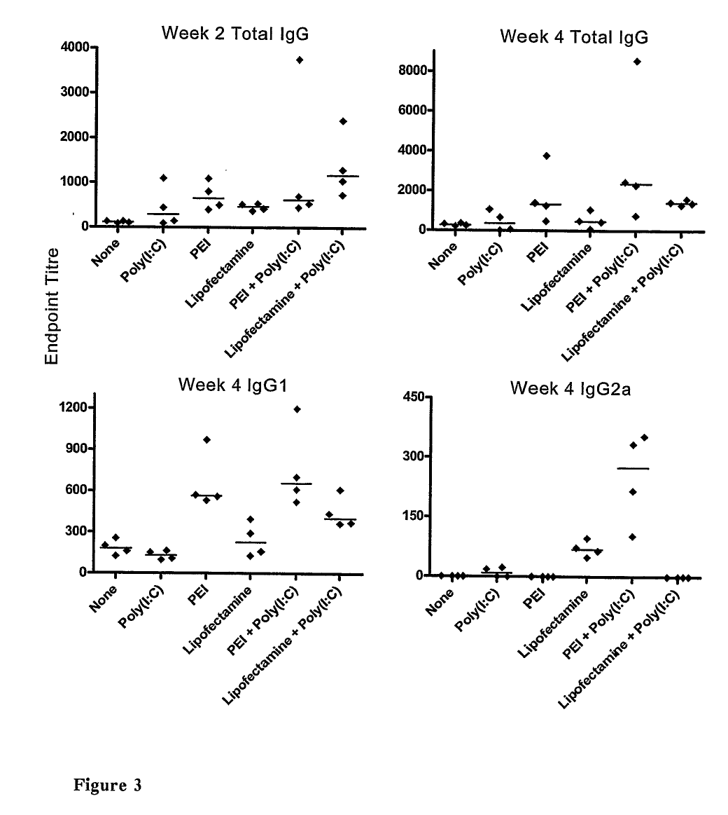

FIG. 3—shows the results of a comparative adjuvant study in mice. More specifically, FIG. 3 illustrates the endpoint antibody titres two or four weeks after a single immunisation for mice immunised with the antigen HIV-1_{CN54}gp140 and an adjuvant comprising the transfection reagent Lipofectamine™ or PEI and/or the TLR ligand Poly(I:C). Control data are also shown.

FIG. 4—shows the nucleotide sequence of a gene which can be used to express the HIV-1_{zm96}gp140 (gp140_{ZM96}) protein. This sequence is the same as Sequence ID No. 1. The entire sequence is that which encodes gp140_{ZM96}, however it can be broken down into: the sequence in normal text—which is the gp120 sequence; and the sequence in italics—which is the gp41 sequence.

FIG. 5—shows the protein sequence of HIV-1_{zm96}gp140 (gp140_{zm96}) protein, as produced by expression of the sequence of FIG. 4. This sequence is the same as Sequence ID No. 3. The sequence in normal text is the gp120 sequence; and the sequence in italics is the gp41 sequence.

FIG. 6—shows the nucleotide sequence of a gene which can be used to express the HIV-1_{CN54}gp140 (gp140_{CN54}) protein. This sequence is the same as Sequence ID No. 2. The entire sequence is that which encodes gp140_{CN54}, however it can be broken down into: the sequence in normal text—which is the gp120 sequence; and the sequence in italics—which is the gp41 sequence;

FIG. 7—shows the protein sequence of HIV-1_{CN54}gp140 (gp140_{CN54}) protein, as produced by expression of the sequence of FIG. 4. This sequence is the same as Sequence ID No. 3. The sequence in normal text is the gp120 sequence; and the sequence in italics is the gp41 sequence.

By immunising rabbits and Balb/c mice with an adjuvant composition comprising a transfection reagent and a candidate vaccine antigen (HIV-1 envelope glycoprotein (Env) gp140 molecules) an increase in antibody response to the antigen was observed.

FIG. 1 shows the results of immunising 4 rabbits subcutaneously with the antigen gp140_{zm96} and the adjuvants Alum, Freunds adjuvant and PEI. Complete Freund's Adjuvant (CFA) is the most powerful adjuvant known; and alum is the standard adjuvant for use in humans. Three groups of four rabbits were immunised subcutaneously with 50 μg of gp140_{ZM96} in the different adjuvants and at weeks 0 and 3 and the serum antibody titres were characterised. As expected, CFA induced the greatest IgG titres to gp140_{zm96}.

The results show that the adjuvant activity of PEI is equivalent to alum, and thus would be a good alternative to alum. In this study uncomplexed PEI was used. The molar dose of alum used was far in excess of the dose of PEI used (64 and 86.2 μmoles/rabbit dose of Al(OH)₃ and Mg(OH)₂ respectively cf 8.1 nmoles of PEI). In addition, the 50:50 mix of antigen with CFA/IFA (Complete Freunds Adjuvant/Incomplete Freunds Adjuvant) gives a much greater dose of CFA/IFA than of PEI. This data shows that much lower doses of PEI can be efficacious as an adjuvant.

The results show that PEI produces a good IgG1 response, indicative of a Th₂ type immune response.

Since some adjuvants are thought to denature protein antigens and thereby favour induction of antibodies to linear rather than conformational epitopes, the ratio of binding antibody titres on native and denatured antigen was studied. The data reveals a trend that CFA and alum are more denaturing than PEI. The ability of an adjuvant to maintain a native conformation of an antigen is important in many cases where antibodies are required to be elicited to properly folded antigens, such as in an HIV Env-based vaccine. This data demonstrates PEI to be a surprisingly better adjuvant than other known adjuvants, as it does not cause significant degradation of the antigen—this will be important when producing vaccines which must generate an immune response capable of recognising the native antigen.

The data presented under the heading “Titre Ratio Native:Denatured” describes the preference of antibodies raised in this study to recognise native versus linearised gp140. The data was obtained by dividing the endpoint IgG titre determined in an ELISA with the native protein by that determined in an ELISA using the denatured protein. The two assays were run together and the median result from 3 independent repeats for each was used to perform this calculation. By presenting the results as a ratio the fold-difference in the binding can readily be seen. For example, a ratio of <1 will occur when the antibody response induced by the antigen+adjuvant combination preferentially recognises the denatured gp140 antigen, whereas a ratio of 1 describes equal binding and anything >1 describes a preference for the native gp140 antigen (i.e. conformationally native rather than denatured linear epitopes). For ¾ rabbits in the PEI group, the ratio approaches 10, suggesting that only a minor component of the antibody response induced recognises linear epitopes. This is in contrast to alum. This data suggests that when alum is used as an adjuvant there is adjuvant-dependent denaturization of the antigen—this would impact on the efficacy of any vaccine using alum. Contrastingly, PEI does not appear to damage the conformation of the antigen.

FIGS. 2 and 3 show the results of immunising 4 groups of mice subcutaneously with 6 μg of the antigen gp140_{CN54} together with different adjuvant combinations. The antibody titres in these mice were characterised in the weeks after the priming immunisation and are plotted in the FIGS. 2 and 3. PEI and FuGENE 6™ are shown to be good adjuvants on their own, stimulating an IgG1 response (a Th₂ response). In the CpG-ODN experiment (FIG. 2) the combination of CpG-ODN with FuGENE 6™ induced an IgG2a response, which was entirely lacking among the other groups after a single immunisation. In the Poly(I:C) experiment (FIG. 3) a combination of Lipofectamine™ and Poly(I:C), or PEI and Poly(I:C) improved the immune response at one or more time points. The combination of PEI and Poly(I:C) induced an IgG2a (Th₁) response that was much superior to the other groups. By controlling the administration of the components a balanced immune response can be produced.

### Methods

gp140_{zm96} Antigen
1. Source Materials of gp140_{zm96} HIV-1 Envelope Protein and Construction of Envelope Expression Plasmids

The C-clade isolate used in the experiments described herein is 96ZM651-8 (Accession no. AF286224) and sequence details are available in (Rodenberg et al, 2001, AIDS Res Hum Retroviruses 17: 161-168). The sequence of the gene used to encode the gp140_{zm96} protein used herein is given in FIG. 4 and Sequence ID no. 1. The sequence of the gp140_{zm96} protein is given in FIG. 5 and Sequence ID no. 2.

The expression system used was the Lonza Biologics (Slough, UK) glutamine synthetase (GS) gene expression system, which has been successfully used to produce correctly folded, fully-functional HIV-1 envelope glycoproteins (gp120, gp140) from Clades A, B, C, D, F and O (Jeffs et al, 1996, G. Gen. Virol. 77: 1403-1410 and Jeffs et al, 2004, Vaccine 22: 1032-1046). The GS vector was pEE14 (available from Celltech Ltd—now UCB), and the cell line was CHO-K1. To maximise gp140 secretion, the signal sequence (ss) of wtgp140 was replaced by that of human tissue plasminogen activator (tpa), a modification that is essential for recombinant HIV-1 envelope glycoprotein secretion from Chinese Hamster Ovary (CHO) cells. To clone the tpa ss and wtgp140 (gp140_{zm96}) gene fragment into pEE14, a pre-existing pEE14/tpa/IIIB gp120 vector was restricted with Bgl II and Eco RI to remove the IIIB gp120 gene, leaving a restricted pEE14/tpa vector into which a gp140_{zm96} gene (obtained from the pCR-Script vector by use of the polymerase chain reaction (PCR) containing a 5′ Bam HI RE site and 3′ Eco RI RE site) is ligated. The DNA sequence of the gp140_{zm96} gene is given in FIG. 4 and Sequence ID no. 1. To facilitate the insertion of this gene, oligonucleotide primers were designed to (a) add Bam HI and Eco RI restriction endonuclease sites to the 5′ and 3′ termini of the ZM96 wtgp140 gene; (b) define the size of the ZM96 wtgp140 gene (commencing at gp120 amino acid G31, and finishing at gp41 amino acid L665; (c) to add a STOP codon immediately downstream of L665. The cleavage site between gp120 and gp41 was modified from REKR to REKS to prevent cleavage.

The terms gp140_{zm96} and ZM96 wtgp140 gene are used interchangeably.

The skilled man will appreciate that the gene of FIG. 4 or Sequence ID No. 1 could be expressed in any suitable expression system to produce the gp140_{zm96} protein.

The skilled man will appreciate that variants of this protein will work in this invention. For example, an alternative leader sequence to the TPA sequence used herein may be used.

In an alternative embodiment the serine amino acid at position 486 may be arginine.

2. Generation of Recombinant CHO Cell Lines with a Stably-Integrated gp140_{ZM96} Gene

Prior to the generation of stable gp140_{zm96} CHO cell lines, the functionality of the pEE14/tpa/ZM96 wtgp140 were ascertained by the use of transient expression assays, using cell-conditioned supernatant (TCSN) from transfected CHO L761H cells as detailed in (Jeffs et al, 2004, Vaccine 22: 1032-1046). Expression levels of gp140_{zm96} were determined by use of a quantitative sandwich ELISA and gp140_{zm96} size/integrity by immunoblots with a rabbit antisera raised against CHO-derived IIIB gp120 (CFAR, NIBSC, UK code ARP422) which detects all expressed recombinant gp120/140/160s tested to date.

The establishment of stable C-clade CHO cell lines follows the protocols previously described (Jeffs et al, 2004, Vaccine 22: 1032-1046), with the modification that at least 100 colonies were selected at an initial input concentration of 25 mM L-methionine sulfoximine (MSX—the selective inhibitor of GS), and the three colonies with the highest specific productivity of trimeric gp140 (as ascertained by reactivity with the human gp41 monoclonal antibody (Mab) 5F3) were cloned by limiting dilution. The highest producing cell lines were then used for bulk production of gp140_{zm96}.

3. Assays for Generation of Stable CHO Cell Lines and in-Process (Bulk Production, Purification) Monitoring

gp140_{zm96} production was monitored by the gp140 quantification assay (Jeffs et al, 2004, Vaccine 22: 1032-1046), during both the generation of stable cell lines and for in-process monitoring. Initially, CHO gp140 from the B-clade isolate IIIB (BH10 clone) (EVA657-CFAR) was used as the standard glycoprotein, but once batches of trimeric ZM96 wtgp140 had been purified this was substituted for IIIB gp140.

4. Bulk Production of gp140_{zm96}

Selected gp140_{zm96} CHO cell lines were used for the bulk production of gp140_{zm96}. Cell lines were used both as adherent cells in serum-containing medium for the small-scale (<1 mg/L) production of gp140_{zm96}, and as suspension-adapted cells in serum-free medium for large-scale (>1 mg/L) gp140_{zm96} production. The initial, adherent, lines were selected and maintained in ExCell™ 302 with 1× GS supplements (JRH Biosciences), supplemented with 5% dialysed foetal bovine serum and 25 mM MSX (Growth Medium). Bulk production was in 850 cm² roller bottles at a starting input of 5×10⁷ cells per bottle in 200 ml of growth medium, TCSN being changed and harvested at 3-4 day intervals.

5. Purification of Trimeric gp140_{zm96}

Trimeric gp140_{zm96} was purified by immunoaffinity chromatography (IAC) using immobilised Mab 5F3. Details of treatment of TCSN from roller bottle harvests prior to IAC is given in (Jeffs et al, 1996, G. Gen. Virol. 77: 1403-1410). 5F3 was linked to beads of AF Tresyl Toyopearl (Tosoh), kindly provided by Dr M-J. Frachette, Aventis Pasteur, Marcy l'Etoile, France. A 5 ml column of 5F3 matrix (5 mg immobilised antibody) was used for each run with clarified TCSN, fractions from each stage of the IAC procedure being monitored for both gp140 and antibody leaching. All runs were carried out at 4° C. at 1 ml/min. Following column equilibration with 5 column volumes (cv) of wash buffer (20 mM Tris-Cl, 500 mM NaCl, 0.01% Triton X-100, pH8.3) and loading of gp140_{zm96} TCSN (max 1 L), non-specifically-bound contaminants were eluted by sequential washing with 5 cv of wash buffer and 10 cv of phosphate buffered saline (PBS-Gibco) pH7.4. 5F3-bound gp140_{zm96} was eluted by elution buffer (50 mM glycine, 500 mM NaCl, pH2.5), each fraction being immediately neutralised by the addition of 4% (v/v) 2M Tris-Cl pH7. Fractions containing gp140 were pooled, concentrated and buffer-exchanged (at least 5 washings) with 20 mM Tris pH7.4 using a 30 Kda molecular weight cut-off microconcentrator (Millipore). If required, further purification was undertaken by size-exclusion chromatography (SEC). An AKTApurifier 10 FPLC system with Unicorn 3.2 software equipped with a Superdex 200 HR 10/300 GL column was used (both GE healthcare), calibrated with marker proteins (thyroglobulin 669 Kda, ferritin 440 Kda, IgG 160 Kda, bovine serum albumin 67 Kda). 5F3-purified batches of gp140_{zm96} (500 mg maximum per run) were injected and separated under native conditions with 50 mM NaPO₄/150 mM NaCl pH7 at a flow rate of 0.5 ml/min. Absorbance was monitored at 280 nm and all treatments and separations were carried out at room temperature. In-process monitoring of IAC and SEC were as follows:—

### IAC

Appearance of TCSN (no contamination/precipitates; colour/turbidity)

A280 (outlet of column)

SDS-PAGE (4-20% Tris-glycine gel)

Immunoblot with anti-Hu/Rb-IgG-HRP (Ab leaching test)

gp140 quantification ELISA with 5F3 (purified IIIB or CN54gp140 as standard)

### SEC

A280 (outlet of column)

Chromatogram

SDS-PAGE (4-20% Tris-glycine gel)

If purity >90%: Protein content (BCA/Bradford—IgG standard)

Immunoblots with 5F3 (trimer), D7324 (monomer), murine gp140 antisera (all)

gp140 quantification ELISA with 5F3 (purified IIIB or CN54gp140 as standard)

6. Characterisation of Purified gp140_{zm96}

IAC (and SEC, if required) purified gp140_{zm96} was fully characterised by reducing and non-reducing PAGE, Immunoblotting and SEC. The antigenicity and functionality of purified gp140_{zm96} was determined by antibody and receptor (CD4, CXCR4) binding using ELISA, immunoblot and FACS-based assays (Jeffs et al, 2004, Vaccine 22: 1032-1046). An initial screen was undertaken with a very wide range of antibodies and a simple “binds strongly” (OD450 value >10× background) or “does not bind” score assigned.

gp140_{CN54} Antigen
1. Source Materials of gp140_{CN54} HIV-1 Envelopes and Construction of Envelope Expression Plasmids

The C-clade isolate used in the experiments described herein is 97CN54 (Accession nos. AX149647 (patent), AF286226 and AF286230) (Su et al, (2000) J. Virol. 74: 11367-11376 and Rodenberg et al, 2001, AIDS Res Hum Retroviruses 17: 161-168). The sequence of the gene used to encode the gp140_{CN54} protein is given in FIG. 6 and Sequence ID No. 2. The protein sequence of gp140_{CN54} protein used in given in FIG. 7 and Sequence ID No. 3.

The expression system used was the Lonza Biologics (Slough, UK) glutamine synthetase (GS) gene expression system, which has been successfully used to produce correctly folded, fully-functional HIV-1 envelope glycoproteins (gp120, gp140) from Clades A, B, C, D, F and O (Jeffs et al, 1996, G. Gen. Virol. 77: 1403-1410 and Jeffs et al, 2004, Vaccine 22: 1032-1046). The GS vector is pEE14 (available from Celltech Ltd, now UCB), and the cell line was CHO-K1. To maximise gp140 secretion, the signal sequence (ss) of wtgp140 was replaced by that of human tissue plasminogen activator (tpa), a modification that is essential for recombinant HIV-1 envelope glycoprotein secretion from Chinese Hamster Ovary (CHO) cells. The syngp140 replaces the wt ss with MNRALLLLLLLLLLLPQAQA. To clone the tpa ss and wtgp140 (gp140_{CN54}) gene fragment into pEE14, a pre-existing pEE14/tpa/IIIB gp120 vector was restricted with Bgl II and Eco RI to remove the IIIB gp120 gene, leaving a restricted pEE14/tpa vector into which a CN54 wtgp140 gene (obtained from the pCR-Script vector by use of the polymerase chain reaction (PCR)) containing a 5′ Bam HI RE site (CN54env contains internal Bgl II RE sites) and 3′ Eco RI RE site was ligated. To facilitate this, oligonucleotide primers were designed to (a) add Bam HI and Eco RI restriction endonuclease sites to the 5′ and 3′ termini of the CN54 wtgp140 gene; (b) define the size of the CN54 wtgp140 gene (commencing at gp120 amino acid G31, and finishing at gp41 amino acid L665; (c) to add a STOP codon immediately downstream of L665. To clone the CN54syngp140 gene (including ss) into pEE14, pEE14 was restricted with Hind III and Eco RI, and oligonucleotide primers and PCR was used to obtain a CN54syngp140 gene from the pCR-Script vector with 5′ Hind III and 3′ Eco RI RE sites, commencing at amino acid M1 and finishing at L665 (plus STOP codon). In both cases the cleavage site between gp120 and gp41 was not modified, thus the expressed glycoproteins are designated CN54 wtgp140REKR and CN54syngp140REKR, and comprise the gp120 subunit domain (less the signal sequence which is cleaved on exit from the CHO cell plasma membrane) and the external domain of gp41 as far as the putative 2F5 epitope (ALDSWKNL).

The skilled man will appreciate that the gene encoding gp140_{CN54} can be cloned and expressed in any suitable expression system to produce the gp140_{CN54} protein.

The terms gp140_{CN54} and CN54 wtgp140 are used interchangeably.

2. Generation of Recombinant CHO Cell Lines with Stably-Integrated C gp140_{CN54} Gene

Prior to the generation of stable gp140_{CN54} CHO cell lines, the functionality of the pEE14/tpa/CN54 wtgp140 was ascertained by the use of transient expression assays, using cell-conditioned supernatant (TCSN) from transfected CHO L761H cells as detailed in (Jeffs et al, 2004, Vaccine 22: 1032-1046). Expression levels of gp140s were determined by use of a quantitative sandwich ELISA and gp140 size/integrity by immunoblots with a rabbit antisera raised against CHO-derived IIIB gp120 (CFAR, NIBSC, UK code ARP422) which detects all expressed recombinant gp120/140/160s tested to date.

The establishment of stable C-clade CHO cell lines follows the protocols previously described (Jeffs et al, 2004, Vaccine 22: 1032-1046), with the modification that at least 100 colonies were selected at an initial input concentration of 25 μM L-methionine sulfoximine (MSX—the selective inhibitor of GS), and the three colonies with the highest specific productivity of trimeric gp140 (as ascertained by reactivity with the human gp41 monoclonal antibody (Mab) 5F3) were cloned by limiting dilution. The highest producing cell lines were then used for bulk production of gp140_{CN54}.

3. Assays for Generation of Stable CHO Cell Lines and in-Process (Bulk Production, Purification) Monitoring

gp140_{CN54} production was monitored by the gp140 quantification assay (Jeffs et al, 2004, Vaccine 22: 1032-1046), during both the generation of stable cell lines and for in-process monitoring. Initially, CHO gp140 from the B-clade isolate IIIB (BH10 clone) (EVA657-CFAR) was used as the standard glycoprotein, but once batches of trimeric CN54 wtgp140REKR had been purified this was substituted for IIIB gp140.

4. Bulk Production of gp140_{CN54}

Selected gp140_{CN54} CHO cell lines were used for the bulk production of gp140_{CN54}. Cell lines were used both as adherent cells in serum-containing medium for the small-scale (<1 mg/L) production of gp140, and as suspension-adapted cells in serum-free medium for large-scale (>1 mg/L) gp140 production. The initial, adherent, lines were selected and maintained in ExCell 302 with 1× GS supplements (JRH Biosciences), supplemented with 5% dialysed foetal bovine serum and 25 μM MSX (Growth Medium). Bulk production was in 850 cm² roller bottles at a starting input of 5×10⁷ cells per bottle in 200 ml of Growth Medium, TCSN being changed and harvested at 3-4 day intervals.

5. Purification of Trimeric gp140_{CN54}

Trimeric gp140_{CN54} was purified by immunoaffinity chromatography (IAC) using immobilised Mab 5F3. Details of treatment of TCSN from roller bottle harvests prior to IAC is given in (Jeffs et al, 1996, G. Gen. Virol. 77: 1403-1410). 5F3 was linked to beads of AF Tresyl Toyopearl (Tosoh), kindly provided by Dr M-J. Frachette, Aventis Pasteur, Marcy l'Etoile, France. A 5 ml column of 5F3 matrix (5 mg immobilised antibody) was used for each run with clarified TCSN, fractions from each stage of the IAC procedure being monitored for both gp140 and antibody leaching. All runs were carried out at 4° C. at 1 ml/min. Following column equilibration with 5 column volumes (cv) of wash buffer (20 mM Tris-Cl, 500 mM NaCl, 0.01% Triton X-100, pH8.3) and loading of gp140_{CN54} TCSN (max 1 L), non-specifically-bound contaminants were eluted by sequential washing with 5 cv of wash buffer and 10 cv of phosphate buffered saline (PBS-Gibco) pH7.4. 5F3-bound gp140_{CN54} was eluted by elution buffer (50 mM glycine, 500 mM NaCl, pH2.5), each fraction being immediately neutralised by the addition of 4% (v/v) 2M Tris-Cl pH7. Fractions containing gp140 were pooled, concentrated and buffer-exchanged (at least 5 washings) with 20 mM Tris pH7.4 using a 30 Kda molecular weight cut-off microconcentrator (Millipore). If required, further purification was undertaken by size-exclusion chromatography (SEC). An AKTApurifier 10 FPLC system with Unicorn 3.2 software equipped with a Superdex 200 HR 10/300 GL column was used (both GE healthcare), calibrated with marker proteins (thyroglobulin 669 Kda, ferritin 440 Kda, IgG 160 Kda, bovine serum albumin 67 Kda). 5F3-purified batches of gp140_{CN54} (500 μg maximum per run) were injected and separated under native conditions with 50 mM NaPO₄/150 mM NaCl pH7 at a flow rate of 0.5 ml/min. Absorbance was monitored at 280 nm and all treatments and separations were carried out at room temperature. In-process monitoring of IAC and SEC were as follows:—

### IAC

Appearance of TCSN (no contamination/precipitates; colour/turbidity)

A280 (outlet of column)

SDS-PAGE (4-20% Tris-glycine gel)

Immunoblot with anti-Hu/Rb-IgG-HRP (Ab leaching test)

gp140 quantification ELISA with 5F3 (purified IIIB or CN54gp140 as standard)

### SEC

A280 (outlet of column)

Chromatogram

SDS-PAGE (4-20% Tris-glycine gel)

If purity >90%: Protein content (BCA/Bradford—IgG standard)

Immunoblots with 5F3 (trimer), D7324 (monomer), murine gp140 antisera (all)

gp140 quantification ELISA with 5F3 (purified IIIB or CN54gp140 as standard)

6. Characterisation of Purified gp140_{CN54}

IAC (and SEC, if required) purified gp140_{CN54} was fully characterised by reducing and non-reducing PAGE, Immunoblotting and SEC. The antigenic topology of purified gp140 was ascertained by antibody and receptor (CD4, CXCR4) binding using ELISA, immunoblot and FACS-based assays (Jeffs et al, 2004, Vaccine 22: 1032-1046). An initial screen was undertaken with a very wide range of antibodies and a simple “binds strongly” (OD450 value >10× background) or “does not bind” score assigned.

### Rabbits

10 to 12 week old, female, New Zealand White rabbits, weighing 2 to 2.5 kg were used.

### Mice

10-12 week old female Balb/c mice were obtained from the specified pathogen-free animal breeding facility at the University of Oxford and housed in micro-isolator cages with filtered air.

All experiments were performed under appropriate licenses in accordance with the UK Animals (Scientific Procedures) Act of 1986.

### Rabbit Immunisations

Buffers were tested for endotoxin at BioManufacturing Facility (Old Road, Headington, Oxford, UK). Rabbits were caged in groups of four by experimental group at the National Institute for Biological Standards and Controls (NIBSC) and each received 50 μg of gp140_{96ZM} in the appropriate adjuvant formulation. Antigen was emulsified with CFA (Sigma) in a 50:50 (v/v) mix (total dose 125 μL per rabbit) by repeatedly drawing the two components through a 19 G needle. For the booster dose, CFA was substituted for Incomplete Freund's Adjuvant (IFA) (Sigma). Alum (40 mg·mL⁻¹ Al(OH)₃, 40 mg·mL⁻¹ Mg(OH)₂) (Pierce) was mixed with the antigen solution 50:50 (total dose 125 μL per rabbit—equivalent to 64 and 86.2 μmoles/rabbit dose of Al(OH)₃ and Mg(OH)₂ respectively) and incubated at room temperature for at least 30 min with frequent agitation to allow antigen precipitation. PEI Linear Sequence average MWt ˜25 KDa (Aldrich Co. Ltd) was diluted stepwise to overcome viscosity to a final concentration of 32.4 μM for immunisation (equivalent to 202.52 μg/rabbit or 8.1 nmoles). The total volume per rabbit was made up to 250 μL (priming immunisation) or 200 μL (booster) as appropriate by addition of sterile, endotoxin-free 5% (w/v) D-glucose (Sigma-Aldrich Co Ltd) 45 min prior to injection. The rabbits were immunised by s.c. (sub cutaneous) injection and monitored for adverse reactions. None of the experimental immunogen preparations used induced ulceration or any other side effects. Blood samples were taken for serological analysis at appropriate time points.

### Mouse Immunisations

Samples of gp140_{CN54} and buffers were tested for endotoxin at The Therapeutic Antibody Centre (Churchill Hospital, University of Oxford, UK). All immunogens were passed through a Detoxi-Gel™ endotoxin affinity column (Perbio, Science UK Ltd, Cramlington, UK) according to manufacturer's instructions in order to minimise endotoxin content. The final concentration of endotoxin in any sample was 0.286EU ml⁻¹ or less. Mice were caged in groups of four by experimental group and each received 6 μg of gp140_{CN54} in the appropriate adjuvant formulation. The prototype murine immunostimulatory CpG-ODN sequence 1018 (underlined, with CpG sequences in bold type): 5′-TGA CTG TGA ACG TTC GAG ATG-3′ has been described elsewhere (Roman et al (2003) J. Immunol 171(6) 3154-3162). GpC-ODN 1018 is a control sequence in which the CpG bases were inverted: 5′-TGA CTG TGA AGC TTG CAG ATG-3′ (MWG Biotech [UK] Ltd, Milton Keynes, UK) and Poly(I:C) (Sigma-Aldrich Co. Ltd, Poole, UK) were used at 100 μg per immunogen dose. The transfection reagent FuGENE 6™ (Roche Diagnostics, Lewes, UK), a blend of lipids and other agents suitable for transfecting mammalian cells in vitro with low toxicity was added to some adjuvant formulations at 6 μl per immunogen dose. PEI Linear Sequence average MWt ˜25 KDa (Sigma-Aldrich Co. Ltd) was diluted stepwise to overcome viscosity to a final concentration of 10 μM for immunisation. Lipofectamine™ reagent (Invitrogen, Paisley, UK) was used at 25 μl (of diluted Lipofectamine™ reagent as supplied by Invitrogen) per vaccine dose. For injection the total volume per mouse was made up to 100 μl as appropriate by addition of sterile, endotoxin-free 20 mM Tris-HCl pH7.4 (Sigma-Aldrich Co Ltd) 45 min prior to injection. The mice were immunised by subcutaneous injection and monitored for adverse reactions. None of the experimental immunogen preparations used induced ulceration or any other side effects. Blood samples were taken for serological analysis at appropriate time points.

### Rabbit ELISAs

High-bind ELISA plates (Greiner Bio-One Ltd, Stonehouse, UK) were coated directly with 50 μL/well of gp140_{ZM96} at a concentration of 0.5 μg·mL⁻¹ in 100 mM NaHCO₃, pH8.5 (Sigma-Aldrich Co Ltd) overnight at 4° C. To test the titre on denatured gp140_{ZM96}, the antigen was diluted to 5 μg·mL⁻¹ in 100 mM NaHCO₃, pH8.5 supplemented with 1% SDS (Sigma) and 50 mM dithiolthreitol (DTT) (Sigma) and heated to 95° C. for 5 min before 10-fold dilution onto the ELISA plates. The plates were washed three times in Dulbecco's phosphate buffered saline (DPBS) (Oxoid, Basingstoke, UK) supplemented with 0.05% Tween 20 (polysorbate 20) (Sigma-Aldrich Co Ltd) and then blocked with 200 μL/well of 2% (w/v) non-fat milk (Marvel) dissolved in DPBS and supplemented with 0.05% Tween 20 for 1 h at RT before the plates were washed as before. A four-fold dilution series of sera ranging from 1:20 to 1:81,920 was prepared in 1% (w/v) BSA (Sigma) dissolved in DPBS (sample buffer) and added directly to the ELISA plate for 1 h. After further washing 50 μL/well of 0.8 μg·mL-1 goat-anti-rabbit-IgG-HRP (Jackson ImmunoResearch Europe Ltd, Soham, UK) secondary Ab diluted in sample buffer was added for 1 h. After a final wash the plates were developed by incubation with 50 μL/well of 1-Step™ ultra-TMB ELISA reagent (Perbio Science UK Ltd, Cramlington, UK) and this reaction was stopped by addition of 50 μL/well 0.5M H₂SO₄ (Sigma). The absorbance was measured at 450 nm. The endpoint titre was determined by calculating the point of intersection between a sigmoidal curve fitted to the titration data and the assay cut-off. The cut-off was calculated as the mean absorbance plus two standard deviations (SD) of wells that lacked serum but were otherwise treated identically. As controls for the native/denatured antigen ELISA, the highly-conformation dependent mAb IgG1b12 and a standard rabbit immune serum (ARP440) was used. The titre on native Env was divided by that on denatured Env to obtain the ratio.

### Mouse ELISAs

High-bind ELISA plates (Greiner Bio-One Ltd, Stonehouse, UK) were coated directly with 50 μl/well of gp140_{CN54} at a concentration of 1 μg/ml⁻¹ in 100 mM NaHCO₃, pH8.5 (Sigma-Aldrich Co Ltd) overnight at 4° C. The plates were washed three times in Dulbecco's phosphate buffered saline [DPBS] (Oxoid, Basingstoke, UK) supplemented with 0.05% Tween 20 [polysorbate 20] (Sigma-Aldrich Co Ltd) and then blocked with 200 μl/well of 2% (w/v) non-fat milk (Marvel™) dissolved in DPBS and supplemented with 0.05% Tween 20 for 1 h at room temperature before the plates were washed as before. A five-fold dilution series of sera ranging from 2.0×10⁻² to 6.4×10⁻⁷ was prepared in 1% (w/v) BSA (Sigma-Aldrich Co Ltd) dissolved in DPBS (sample buffer) and added directly to the ELISA plate for 1 h. After further washing 50 μl/well of 0.8 μg/ml⁻¹ rabbit-anti-mouse-IgG-HRP (Jackson ImmunoResearch Europe Ltd, Soham, UK) or rat-anti-mouse-IgG1 or IgG2a-HRP (BD Biosciences Pharmingen, Oxford, UK) secondary Ab diluted in sample buffer was added for 1 h. After a final wash the plates were developed by incubation with 50 μl/well of 1-Step™ ultra-TMB ELISA reagent (Perbio Science UK Ltd, Cramlington, UK) and this reaction was stopped by addition of 50 μl/well 0.5M H2SO4 (Sigma-Aldrich Co Ltd). The absorbance was measured at 450 nm. The endpoint titre was determined by calculating the serum dilution at which the line of best fit to the linear portion of the curve bisected the assay cut-off. The cut-off was calculated as the mean absorbance plus two standard deviations (SD) of wells that lacked mouse serum but were otherwise treated identically.

### Statistics on Rabbit Data

The rabbit ELISA data was analysed in GraphPad Prism (version 4.02 for Windows, GraphPad Software, San Diego Calif. USA, www.graphpad.com). The endpoint antibody titre was calculated for each rabbit on 3-4 independent occasions. The median titre obtained for each animal was then plotted and the group median titre shown graphically. Multiple groups were compared by Kruskal-Wallis analysis with the Dunn's Multicomparison test applied where the Kruskal-Wallis test gave P<0.05. The Dunn's Test reveals groups that are largely responsible for any significant result from the Kruskal-Wallis test. For post hoc analysis of selected pairs of groups, a two-tailed Mann Whitney test was used to determine whether increases in titre were significant between pairs. Untested pairs were clearly not significant by examination of the graphs.

FIG. 1 shows

### Prime Total IgG

### Kruskal-Wallis: P=0.0264

Mann Whitney test: Alum Vs PEI: P=0.3429 (not significant)

### Prime Native:Denatured Ratio

Kruskal-Wallis: P=0.0627 (not significant)

### Boost Total IgG

### Kruskal-Wallis: P=0.0125

Mann Whitney test: Alum Vs PEI: P=0.1143 (not significant)

### Boost Native:Denatured Ratio

Kruskal-Wallis: P=0.08 (not significant)

### Statistics on Mouse Data

The ELISA data was analysed in GraphPad Prism (version 4.02 for Windows, GraphPad Software, San Diego Calif. USA, www.graphpad.com). The endpoint antibody titre was calculated for each mouse on 3-4 independent occasions. The median titre obtained for each mouse was then plotted and the group median titre shown graphically. Multiple groups were compared by Kruskal-Wallis analysis with the Dunn's Multicomparison test applied where the Kruskal-Wallis test gave P<0.05. The Dunn's Test reveals groups that are largely responsible for any significant result from the Kruskal-Wallis test. For post hoc analysis of selected pairs of groups (an adjuvant combination and its constituent parts) a one-tailed Mann Whitney test was used to determine whether increases in titre were significant between pairs. Untested pairs were clearly not significant by examination of the graphs.

Results of less than P=0.05 were considered significant.

FIG. 2 shows the endpoint antibody titres two or five weeks after a single immunisation for mice immunised with the antigen HIV-1 gp140CN54, the TLR ligand CpG-ODN and the transfection reagent FuGENE 6™, control data are also shown.

By looking at IgG1 levels an indication of the Th2 response in a subject can be determined. By looking at IgG2a levels an indication of the Th1 response in a subject can be determined.

The results of the most relevant statistical analysis is given below.

Week 2 total IgG
Kruskal-Wallis: P=0.0685 (not significant)—this result indicates that any differences between the different groups are not statistically significant.
Week 5 total IgG

### Kruskal-Wallis: P=0.0274

Mann Whitney test: FuGENE 6™ vs. FuGENE 6™+CpG: P=0.0571 (not significant). However the difference between FuGENE 6™+CpG and FuGENE 6™+GpC is clearly significant.

### Week 5 IgG1

### Kruskal-Wallis: P=0.033

### Week 5 IgG2a

Kruskal-Wallis: P=0.0004—this result indicates that the effect seen with FuGENE 6™+CpG is highly statistically significant.

The above statistical analysis and FIG. 2 show that the combination of FuGENE 6™ and CpG induced an IgG2a (Th1) response, which was absent in the other groups. The combination of FuGENE 6™ and CpG also achieved a total IgG median titre twice as high as the next group, FuGENE 6™ alone, however this difference does not quite reach significance (P=0.0571), suggesting that the group size was not large enough to determine the significance of the difference. However, it is clear that the combination affected the immune response as indicated by the IgG2a response.

FIG. 3 shows the endpoint titres two or four weeks after a single immunisation for mice immunised with the antigen HIV-1 gp140_{CN54}, the TLR ligand Poly(I:C) and the transfection reagent Lipofectamine™ or PEI. Control data are also shown.

### Week 2 Total IgG

PEI—Not significant

### Lipofectamine™—Not Significant

### Week 4 Total IgG

PEI—Not significant.

### Lipofectamine™

Kruskall-Wallis test:

### Poly(I:C), Lipofectamine™ and Poly(I:C)+Lipofectamine™, P=0.0244

One-tailed Mann-Whitney test with Bonferroni's Correction:

### Poly(I:C)+Lipofectamine™>Poly(I:C), P=0.0143

### Poly(I:C)+Lipofectamine™>Lipofectamine™, P=0.0143

### Week 4 IgG1

### PEI

Kruskall-Wallis test:

### Poly(I:C), PEI and Poly(I:C)+PEI, P=0.0243

One-tailed Mann-Whitney test with Bonferroni's Correction:

### Poly(I:C)+PEI>Poly(I:C), P=0.0143

Poly(I:C)+PEI>PEI, P=0.3429, not significant

### Lipofectamine™

Kruskall-Wallis test:

### Poly(I:C), Lipofectamine™ and Poly(I:C)+Lipofectamine™, P=0.0296

One-tailed Mann-Whitney test with Bonferroni's Correction:

### Poly(I:C)+Lipofectamine™>Poly(I:C), P=0.0143

Poly(I:C)+Lipofectamine™>Lipofectamine™, P=0.0571, not significant

### Week 4 IgG2a

### PEI

Kruskall-Wallis test:

### Poly(I:C), PEI and Poly(I:C)+PEI, P=0.0105

One-tailed Mann-Whitney test with Bonferroni's Correction:

### Poly(I:C)+PEI>Poly(I:C), P=0.0143

Poly(I:C)+PEI>PEI, P<0.01 significant (no response with PEI alone so test not applicable)

### Lipofectamine™

Kruskall-Wallis test:
Poly(I:C), Lipofectamine™ and Poly(I:C)+Lipofectamine™, P=0.0105 Lipofectamine™ alone is the only group with a measurable response.

The above statistics and FIG. 3 show that PEI alone, PEI and Poly(I:C), and Lipofectamine™ and Poly(I:C) were effective adjuvants. The combination of PEI+Poly(I:C) induced the greatest IgG2a (Th1) response despite PEI alone inducing a strongly Th2 response, and Poly(I:C) alone being ineffective as an adjuvant.

The results in FIGS. 2 and 3 show that ligands for TLR3 and TLR9, namely CpG-ODN and Poly(I:C), function more effectively as adjuvants to induce antibody responses when they are combined with transfection reagents. CpG-ODN were tested with a single transfection reagent, FuGENE 6™, while Poly(I:C) was tested in combination with Lipofectamine™ and PEI. Specifically, IgG2a responses to gp140_{CN54} could be induced in the presence of a combination of FuGENE 6™ and CpG-ODN, or PEI and Poly(I:C), while such responses were absent when the components were tested alone. Median total IgG responses were highest for the combinations of PEI and Poly(I:C), and FuGENE 6™ and CpG-ODN at week 4/5.

1. (canceled)

2. An adjuvant composition comprising a transfection reagent.

3. The composition of claim 2 wherein the transfection reagent is non-liposomal.

4. The composition of claim 2 wherein the non-liposomal transfection reagent is a cationic polymer.

5. The composition of claim 2 wherein the transfection reagent is PEI or an effective derivative of PEI.

6. The composition of claim 2 wherein the adjuvant stimulates an immune response selected from a Th₁ immune response, a Th₂ immune response and a combination of a Th₁ and a Th₂ immune response, when administered to a human or non-human animal.

7. The composition of claim 2 wherein the adjuvant comprises one or more ligands for one or more intracellular immune response receptors.

8. The composition of claim 2 wherein the adjuvant does not comprise a ligand for one or more intracellular immune response receptors.

9. An immunogenic composition capable of eliciting an immune response to an antigen when administered to a human or non-human animal comprising one or more antigens and an adjuvant composition comprising a transfection reagent.

10. The composition of claim 9 for use as a vaccine.

11. (canceled)

12. The composition of claim 9 wherein the antigen is selected from the group comprising a nucleic acid, a protein, a peptide, a glycoprotein, a polysaccharide, a carbohydrate, a fusion protein, a lipid, a glycolipid, a peptide mimic of a polysaccharide, a cell, a cell extract, a dead or attenuated cell or extract thereof, a tumour cell or an extract thereof, or a viral particle or an extract thereof, and any combination thereof.

13. The composition of claim 9 wherein the antigen is derived from a human or non-human animal, a bacterium, a virus, a fungus, a protozoan or a prion.

14. The composition claim 9 wherein the antigen is derived from a pathogen.

15. The composition of claim 12 wherein the antigen is a protein or polypeptide derived from one or more of the following pathogens, HIV type 1, HIV type 2, the Human T Cell Leukaemia Virus type 1, the Human T Cell Leukaemia Virus type 2, the Herpes Simplex Virus type 1, the Herpes Simplex Virus type 2, the human papilloma virus, Treponema pallidum, Neisseria gonorrhoea, Chlamydia trachomatis and Candida albicans.

16. The composition of claim 9 wherein the antigen is an HIV envelope glycoprotein (Env), or a fragment or an immunogenic derivative thereof.

17. The composition of claim 9 wherein the antigen has at least 65% identity to the sequence of Sequence ID no: 2 or 4.

18. The composition of claim 2 for use in therapeutic or prophylactic treatments.

19. (canceled)

20. (canceled)

21. (canceled)

22. (canceled)

23. (canceled)

24. (canceled)

25. A method for inducing or enhancing immunogenicity of an antigen in a human or non-human animal subject to be treated, comprising administering to said subject one or more antigens and an adjuvant composition according to claim 2 in an amount effective to induce or enhance the immunogenicity of the antigen in the subject.

26. The method of claim 25 wherein the adjuvant and antigen are administered simultaneously, sequentially or separately.

27. The method of claim 17 wherein the immune reaction produced is sufficient to vaccinate a subject against a pathogen from which the antigen is derived.
